(19) United States
(12) Patent Application Publication
(10) Pub. No.: US 2023/0363656 A1
(43) Pub. Date: Nov. 16, 2023

(54) BLOOD PRESSURE MEASURING METHOD AND A BLOOD PRESSURE MEASURING SYSYEM

(71) Applicant: National Taiwan University Of Science And Technology, Taipei City (TW)

(72) Inventors: CHAO-HSIUNG TSENG, Taipei City (TW); SHI-WEI WU, Taipei City (TW)

(21) Appl. No.: 18/088,027

(22) Filed: Dec. 23, 2022

(30) Foreign Application Priority Data
May 11, 2022 (TW) 111117719

(51) Int. Cl. A61B 5/024; A61B 5/00; A61B 5/021

(52) U.S. Cl. CPC A61B 5/02416; A61B 5/725; A61B 5/7257; A61B 5/02108; A61B 5/681

(57) ABSTRACT

A blood pressure measuring method includes the following steps: obtaining a plurality of infrared physiological signals from a wrist radial artery of a user by a physiology signal sensor; processing the plurality of infrared physiological signals for obtaining a plurality of single pulse signals by a signal processing mechanism; performing Fourier expansion on each of the plurality of single pulse signals for extracting a characteristic of each of the plurality of single pulse signals; and inputting the characteristic of each of the plurality of single pulse signals to a blood pressure measuring model established by a convolutional neural network for obtaining a systolic blood pressure and a diastolic blood pressure according to the plurality of infrared single pulse signals.

### BACKGROUND OF THE INVENTION

### 1. Field of the Invention

The present invention relates to a blood pressure measuring method and a blood pressure measuring system; more particularly, the present invention relates to a blood pressure measuring method and a blood pressure measuring system utilizing infrared single pulse signals.

### 2. Description of the Related Art

With the booming development of physiological information technology related to smart wearable devices, a user wearing the smart wearable device can at any time monitor his/her physiological indices, such as the respiration rate, the pulse rate, the temperature, or the like. However, because the smart wearable device is not a medical instrument, it can only provide a blood pressure reference value instead of an actual blood pressure value. Further, if there is a need for measuring the blood pressure via the smart wearable device, it is required to have the user's finger directly contact the metal contact of the smart wearable device. As a result, the user cannot monitor the blood pressure at any time, which limits the usage scope of the smart wearable device. Moreover, the price of such a smart wearable device equipped with a blood pressure sensor is high.

Therefore, there is a need to provide a blood pressure measuring method and a blood pressure measuring system to mitigate and/or obviate the aforementioned problems.

### SUMMARY OF THE INVENTION

It is an object of the present invention to provide a blood pressure measuring method utilizing infrared single pulse signals.

It is another object of the present invention to provide a blood pressure measuring system utilizing infrared single pulse signals.

To achieve the abovementioned objects, the blood pressure measuring method of the present invention includes the following steps: obtaining a plurality of infrared physiological signals from a wrist radial artery of a user via a physiology signal sensor; processing the plurality of infrared physiological signals for obtaining a plurality of infrared single pulse signals via a signal processing mechanism; performing Fourier expansion on each of the plurality of infrared single pulse signals for extracting a characteristic of each of the plurality of infrared single pulse signals; and inputting the plurality of extracted characteristics into a blood pressure measuring model established by a convolutional neural network (CNN) for obtaining a systolic blood pressure value or a diastolic blood pressure value corresponding to the plurality of infrared single pulse signals.

The present invention further provides a blood pressure measuring system comprising a physiology signal sensor, a signal processing module and a calculation module. The physiology signal sensor is used for obtaining a plurality of infrared physiological signals from a wrist radial artery of a user. The signal processing module has a signal connection with the physiology signal sensor. The signal processing module processes the plurality of infrared physiological signals for obtaining a plurality of infrared single pulse signals via a signal processing mechanism and performs Fourier expansion on each of the plurality of infrared single pulse signals for extracting a characteristic of each of the plurality of infrared single pulse signals. The calculation module has a signal connection with the signal processing module. The calculation module is used for obtaining a systolic blood pressure value or a diastolic blood pressure value corresponding to the plurality of infrared single pulse signals by means of inputting the plurality of extracted characteristics into a blood pressure measuring model as disclosed in the abovementioned blood pressure measuring method.

In the blood pressure measuring method and the blood pressure measuring system of the present invention, after noise and respiration signals are filtered out of the plurality of infrared physiological signals, a plurality of infrared single pulse signals will be obtained. The invention then performs Fourier expansion on each of the plurality of infrared single pulse signals for obtaining the extracted characteristics, including sine coefficients and cosine coefficients extracted from each of the plurality of infrared single pulse signals, and inputting those extracted characteristics into the blood pressure measuring model established by the CNN so as to obtain an actual blood pressure value corresponding to the plurality of infrared single pulse signals. Further, training the blood pressure measuring model by extracting the characteristics from the infrared single pulse signals does not require a large amount of training data, which means a small amount of training data is sufficient for training. Therefore, it is easier to accomplish a clinical trial for the blood pressure measuring model used in the blood pressure measuring method of the present invention.

Other objects, advantages, and novel features of the invention will become more apparent from the following detailed description when taken in conjunction with the accompanying drawings.

### BRIEF DESCRIPTION OF THE DRAWINGS

These and other objects and advantages of the present invention will become apparent from the following description of the accompanying drawings, which disclose several embodiments of the present invention. It is to be understood that the drawings are to be used for purposes of illustration only, and not as a definition of the invention.

In the drawings, wherein similar reference numerals denote similar elements throughout the several views:

FIG. 1A illustrates a schematic drawing showing a blood pressure measuring system in use according to the present invention.

FIG. 1B illustrates a hardware structural drawing of the blood pressure measuring system according to the present invention.

FIG. 2 illustrates a flowchart of a blood pressure measuring method according to the present invention.

FIG. 3 illustrates a flowchart of a signal processing mechanism of the blood pressure measuring method according to the present invention.

FIG. 4 illustrates a schematic drawing showing the plurality of infrared single pulse signals after being processed by the signal processing mechanism of the blood pressure measuring method according to the present invention.

FIG. 5 illustrates a schematic drawing showing an infrared single pulse signal and the infrared single pulse signal rebuilt by utilizing the plurality of extracted characteristics of the infrared single pulse signal.

FIG. 6 illustrates a schematic drawing showing the waveform characteristics of the infrared single pulse signal synthesized by utilizing the extracted characteristics at multiple different frequencies.

FIG. 7 illustrates a flowchart of computation steps of a blood pressure measuring model of the blood pressure measuring method according to the present invention.

FIG. 8 illustrates a plot showing predicted systolic blood pressure values and reference systolic blood pressure values calculated by the blood pressure measuring model of the blood pressure measuring method according to the present invention.

FIG. 9 illustrates a plot showing predicted diastolic blood pressure values and reference diastolic blood pressure values calculated by the blood pressure measuring model of the blood pressure measuring method according to the present invention.

### DETAILED DESCRIPTION OF THE PREFERRED EMBODIMENT

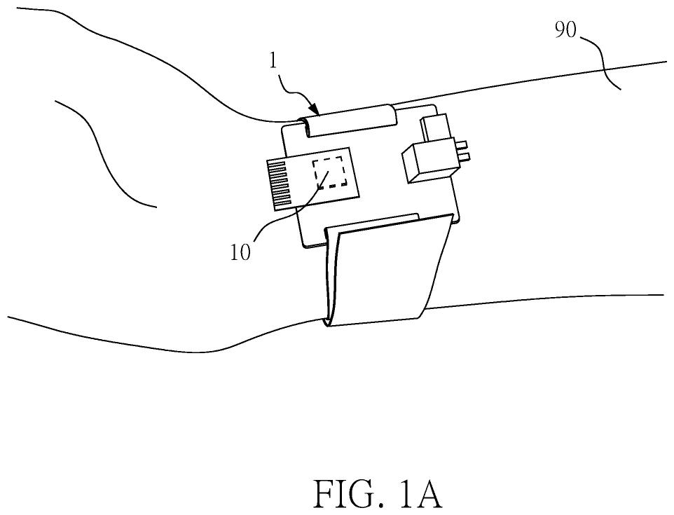

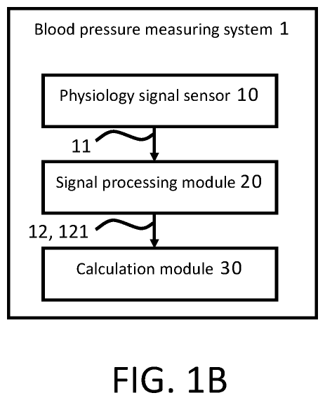

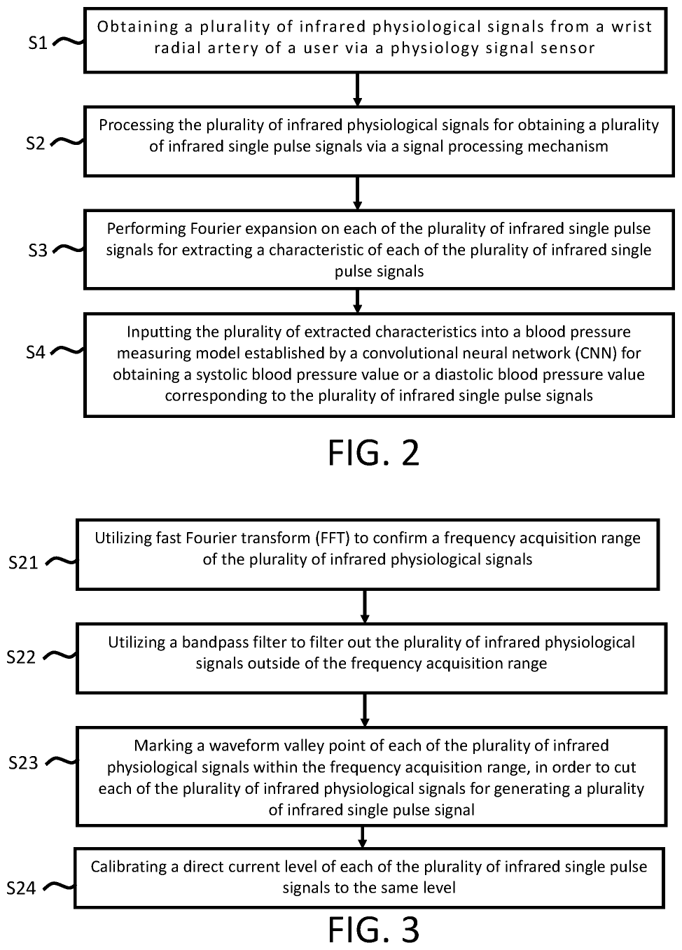

Please refer to FIG. 1A, FIG. 1B and FIG. 2. FIG. 1 illustrates a schematic drawing showing a blood pressure measuring system in use according to the present invention; FIG. 1B illustrates a hardware structural drawing of the blood pressure measuring system according to the present invention; and FIG. 2 illustrates a flowchart of a blood pressure measuring method according to the present invention.

As shown in FIG. 1A, FIG. 1B and FIG. 2, the blood pressure measuring method of the present invention is used in a blood pressure measuring system 1. The blood pressure measuring system 1 of the present invention can be installed in a smart wearable device or implemented as an independent medical instrument. The blood pressure measuring system 1 comprises a physiology signal sensor 10, a signal processing module 20 and a calculation module 30. The physiology signal sensor 10 is placed on a wrist radial artery of a user 90 for obtaining a plurality of infrared physiological signals 11. In this embodiment, the physiology signal sensor 10 is, but is not limited to, a photoplethysmography sensor (PPG sensor); the physiology signal sensor 10 can be other equivalent photosensors capable of detecting physiological signals. The signal processing module 20 has a signal connection with the physiology signal sensor 10. The signal processing module 20 is used for processing the plurality of infrared physiological signals 11 for obtaining a plurality of infrared single pulse signals 12 via a signal processing mechanism and for performing Fourier expansion on each of the plurality of infrared single pulse signals 12 for extracting a characteristic 121 of each of the plurality of infrared single pulse signals 12. In this embodiment, the plurality of extracted characteristics 121 include sine coefficients and cosine coefficients extracted from each of the plurality of infrared single pulse signals 12 after Fourier expansion. The calculation module 30 has a signal connection with the signal processing module 20. The calculation module 30 is used for obtaining a systolic blood pressure value or a diastolic blood pressure value corresponding to the plurality of infrared physiological signals 11 based on the plurality of extracted characteristics 121 and the blood pressure measuring method according to the present invention.

Please note that the operations of the signal processing mechanism and the blood pressure measuring model of the blood pressure measuring system 1 of the present invention will be explained in subsequent related paragraphs corresponding to the blood pressure measuring method of the present invention. Furthermore, the signal processing module 20 and the calculation module 30 of the blood pressure measuring system 1 of the present invention can be configured not only as hardware devices, software programs, firmware or combinations thereof but also as a circuit loop or other applicable forms. Moreover, each of the modules can be configured in an independent form or a joint form. In one preferred embodiment, each module is a software program stored in a memory, and a processor (not shown in figures) of the blood pressure measuring system 1 will run the signal processing module 20 and the calculation module 30 in order to achieve the object of the present invention. Further, the embodiments described herein are only preferred embodiments of the present invention. To avoid redundant description, not all possible variations and combinations are described in detail in this specification. However, those skilled in the art will understand that the above modules or components are not all necessary parts and that, in order to implement the present invention, other more detailed known modules or components might also be included. It is possible that each module or component can be omitted or modified depending on different requirements, and it is also possible that other modules or components might be disposed between any two modules.

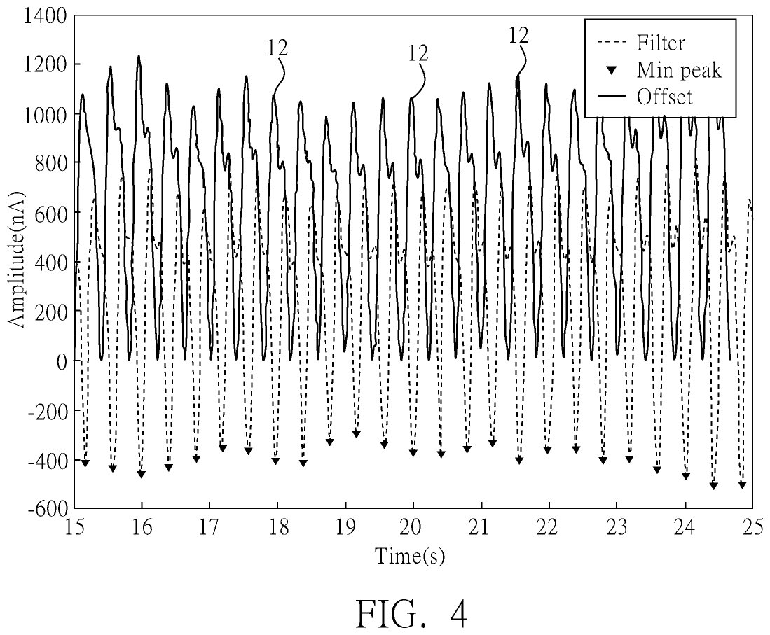

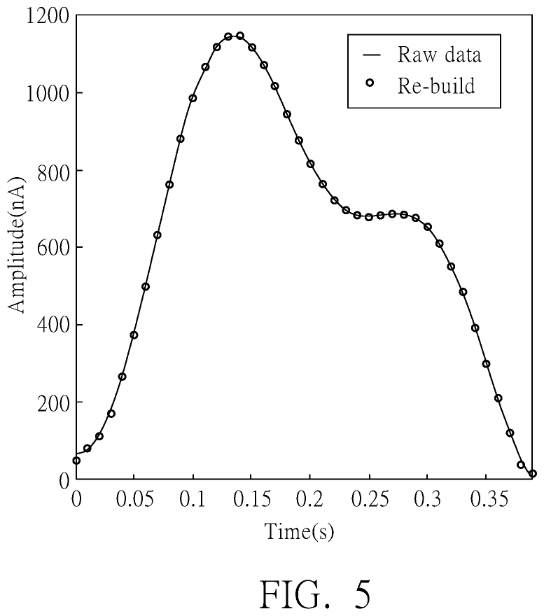

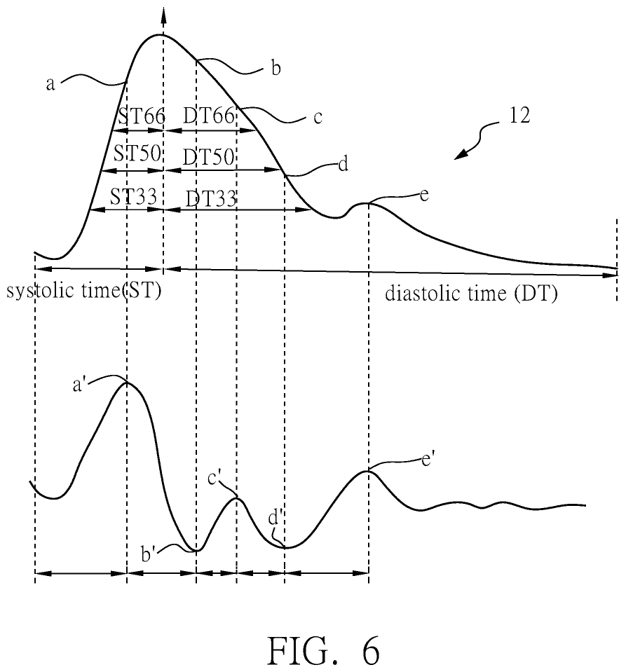

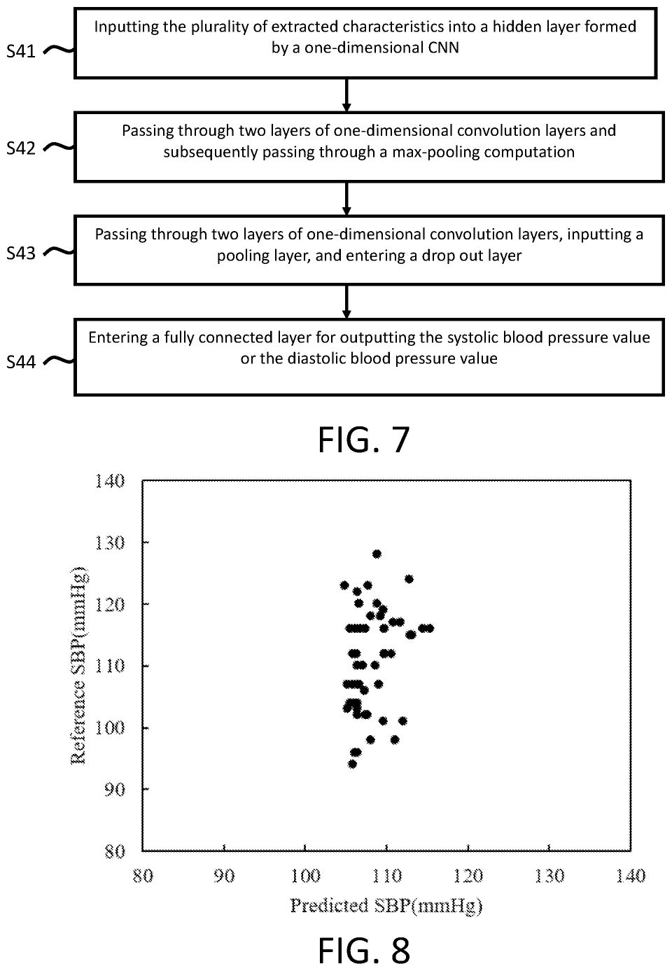

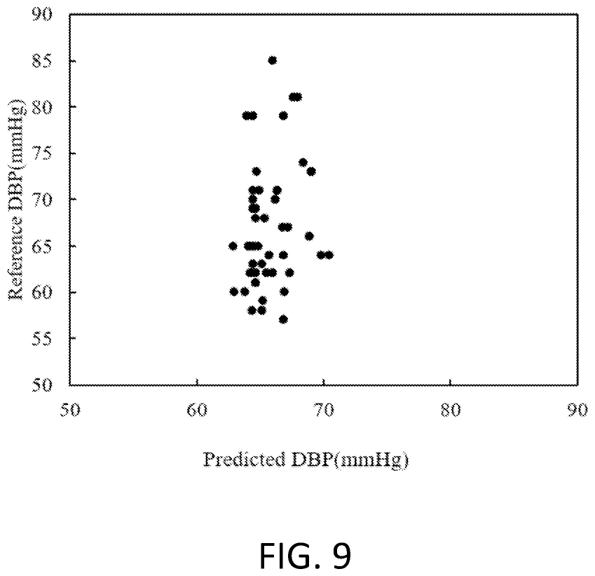

Please refer to FIG. 1A and FIG. 1B, and together refer to FIG. 2 to FIG. 9, wherein FIG. 2 illustrates a flowchart of a blood pressure measuring method according to the present invention; FIG. 3 illustrates a flowchart of a signal processing mechanism of the blood pressure measuring method according to the present invention; FIG. 4 illustrates a schematic drawing showing the plurality of infrared single pulse signals after being processed by the signal processing mechanism of the blood pressure measuring method according to the present invention; FIG. 5 illustrates a schematic drawing showing an infrared single pulse signal and the infrared single pulse signal rebuilt by utilizing the plurality of extracted characteristics of the infrared single pulse signal; FIG. 6 illustrates a schematic drawing showing the waveform characteristics of the infrared single pulse signal synthesized by utilizing the extracted characteristics at multiple different frequencies; FIG. 7 illustrates a flowchart of computation steps of a blood pressure measuring model of the blood pressure measuring method according to the present invention; FIG. 8 illustrates a plot showing predicted systolic blood pressure values and reference systolic blood pressure values calculated by the blood pressure measuring model of the blood pressure measuring method according to the present invention; and FIG. 9 illustrates a plot showing predicted diastolic blood pressure values and reference diastolic blood pressure values calculated by the blood pressure measuring model of the blood pressure measuring method according to the present invention.

As shown in FIG. 2, the blood pressure measuring method of the present invention is used in a blood pressure measuring system 1 and comprises steps Si to S4. Please refer to the following paragraphs for descriptions of each step of the blood pressure measuring method of the present invention in detail.

Step S1: obtaining a plurality of infrared physiological signals from a wrist radial artery of a user via a physiology signal sensor.

In the blood pressure measuring method of the present invention, a physiology signal sensor is placed on a wrist radial artery of a user for obtaining a plurality of infrared physiological signals. In this embodiment, the physiology signal sensor is, but is not limited to, a photoplethysmography sensor (PPG sensor); however, the physiology signal sensor can be other equivalent photosensors capable of detecting physiological signals.

Step S2: processing the plurality of infrared physiological signals for obtaining a plurality of infrared single pulse signals via a signal processing mechanism.

Because the plurality of infrared physiological signals also include high frequency noises, low frequency noises and the users' respiration signals, the present invention utilizes the signal processing mechanism to filter out the abovementioned noises in order to retain only the plurality of infrared single pulse signals in the plurality of infrared physiological signals. As shown in FIG. 2, in this embodiment, the signal processing mechanism comprises steps S21 to S24 described in detail below.

Step S21: utilizing fast Fourier transform (FFT) to confirm a frequency acquisition range of the plurality of infrared physiological signals.

First, FFT is utilized to observe the spectrum of the plurality of infrared physiological signals. Because the plurality of infrared physiological signals obtained from the physiology signal sensor include both respiration physiological signals and pulse physiological signals, in order to prevent the process of filtering out the noises from the plurality of infrared physiological signals from being affected by other frequency multiplication or the DC offset voltage, FFT is utilized in this embodiment to confirm the frequency acquisition range of the plurality of infrared physiological signals; that is, FFT is utilized to retain the frequency range of the pulse physiological signals.

Step S22: utilizing a bandpass filter to filter out the plurality of infrared physiological signals outside of the frequency acquisition range.

In this embodiment, the bandpass filter is an IIR Chebyshev filter type 2, which is used as a digital filter to filter out high frequency noises, low frequency noises and the user's respiration signals from the plurality of infrared physiological signals. The requirements for the high pass cutoff frequency and the low pass cutoff frequency are different; therefore, for practicality in this embodiment, the plurality of infrared physiological signals pass through a high pass filter first and then through a low pass filter so that high frequency noises, low frequency noises and the respiration signals of the user 90 can be filtered out of the plurality of infrared physiological signals. In this embodiment, the high pass filter for the plurality of infrared physiological signals has a stop band cutoff frequency of 0.3 Hz and a pass band cutoff frequency of 0.5 Hz, and the low pass filter for the plurality of infrared physiological signals has a pass band cutoff frequency of 6 Hz and a stop band cutoff frequency of 6.5 Hz. Therefore, the bandpass filter is formed accordingly for filtering out the plurality of infrared physiological signals outside of the frequency acquisition range.

Step S23: marking a waveform valley point of each of the plurality of infrared physiological signals within the frequency acquisition range, in order to cut each of the plurality of infrared physiological signals for generating a plurality of infrared single pulse signals.

After the plurality of infrared physiological signals 11 respectively pass through the high pass filter and the low pass filter of the bandpass filter, a plurality of infrared pulse signals will remain. Due to the non-ideal characteristic of the filter selected in this embodiment, the first 1,500 points of the plurality of infrared single pulse signals 12 may comprise oscillation. As a result, in this embodiment, the data after 1,500 points will be selected; that is, the plurality of infrared physiological signals obtained from the physiology signal sensor about 15 seconds after the sensor begins to work will be selected. As shown in FIG. 4, the dotted lines refer to the signals passing through the filter. The present invention will find the valley point of each of the plurality of infrared single pulse signals 12. In this embodiment, the inverted triangles in FIG. 4 indicate the valley points of each of the plurality of infrared single pulse signals 12. The indexes of the valley points of each of the plurality of infrared single pulse signals are recorded, the first and the last incomplete infrared single pulse signals 12 are removed, and the index is utilized to cut each of the plurality of infrared physiological signals to form a plurality of independent infrared single pulse signals. Each infrared single pulse signal represents each heartbeat; as a result, even though each of the plurality of infrared single pulse signal looks similar to the others, they are all independent signals.

Step S24: calibrating a direct current (dc) level of each of the plurality of infrared single pulse signals to the same level.

As shown in FIG. 4, after the filtering process, there are 23 complete infrared single pulse signals 12, and each of the infrared single pulse signals 12 is cut out. Because each of the cut infrared single pulse signals 12 has a different dc level, the dc level of each of the infrared single pulse signals 12 must be calibrated to the same level, and thus the present invention can then perform step S3 to perform characteristic extraction to the infrared single pulse signals 12.

Step S3: performing Fourier expansion on each of the plurality of infrared single pulse signals for extracting a characteristic of each of the plurality of infrared single pulse signals.

In general, human physiological signals, such as respiration and heartbeat signals, are periodic signals. Theoretically, these periodic signals can be represented by sine coefficients and cosine coefficients. However, in reality, such periodic phenomena are complicated. Complicated functions can be defined by performing linear combination to sine functions and cosine functions at different frequencies; therefore, the present invention performs Fourier expansion on these periodic signals (according to the following Formula 1), wherein aₖ and bₖ defined in Formula 1 can be calculated according to the following Formula 2 and Formula 3. If the periodic signals turn into a discrete form, the formulas will need to be revised as Formulas 4, 5 and 6 provided below, where k is the number of harmonic waves, n is the index of the data point, and N is the sum of total data points of the cut waveform (i.e., the cut independent infrared single pulse signal).

$$f ( x ) = \frac{1}{2} {a}_{0} + {\sum }_{k = 1}^{∞} [ {a}_{k} cos \frac{2 π kx}{L} + {b}_{k} sin \frac{2 π kx}{L} ] \qquad ( Formula 1 )$$

$${a}_{k} = \frac{2}{L} {\int }_{- L / 2}^{L / 2} f ( x ) cos \frac{2 π kx}{L} dx ( k = 0 , 1 , 2 , … ) \qquad ( Formula 2 )$$

$${b}_{k} = \frac{2}{L} {\int }_{- L / 2}^{L / 2} f ( x ) sin \frac{2 π kx}{L} dx ( k = 1 , 2 , 3 , … ) \qquad ( Formula 3 )$$

$$x [ n ] = \frac{1}{2} {a}_{0} + {\sum }_{k = 1}^{M} [ {a}_{k} cos \frac{2 π kn}{N} + {b}_{k} sin \frac{2 π kn}{N} ] \qquad ( Formula 4 )$$

$${a}_{k} = \frac{2}{N} {\sum }_{n = 1}^{N - 1} x [ n ] cos \frac{2 π kn}{N} ( k = 0 , 1 , 2 , … , M ) \qquad ( Formula 5 )$$

$${b}_{k} = \frac{2}{N} {\sum }_{n = 1}^{N - 1} x [ n ] sin \frac{2 π kn}{N} ( k = 1 , 2 , 3 , … , M ) \qquad ( Formula 6 )$$

In this invention, calibration is performed to adjust the direct current (dc) level of each of the plurality of cut infrared single pulse signals 12 depicted as solid lines in FIG. 4; then Fourier expansion is performed on the plurality of adjusted infrared single pulse signals 12 for obtaining the Fourier series of the plurality of infrared single pulse signals 12. Fourier transform is adopted here because according to the above Formula 4, it is necessary to know aₖ and bₖ before obtaining xᵢ, and aₖ and bₖ can be obtained according to the above Formula 5 and Formula 6, which are equivalent to the real part and the imaginary part obtained by Fourier transform. Therefore, fast Fourier transform (FFT) is performed to obtain aₖ and bₖ, which represent the real part and the imaginary part of Fourier transform of each of the cut infrared single pulse signals 12, as well as the sine coefficients and the cosine coefficients; i.e., the plurality of extracted characteristics of the present invention. After the orthogonal basis of the Fourier series is obtained, the plurality of extracted characteristics of twelve (12) harmonic waves of the twelve (12) selected infrared single pulse signals 12, i.e., the sine coefficients and cosine coefficients of the single waveform Fourier transform after being cut, are represented as the following Table 1:

TABLE 1
Training data saving orders

| | |
|---|---|
| a0 | 2480 |
| a1 | 7880 |
| b1 | −3509 |
| a2 | −3954 |
| b2 | 1750 |
| . | . |
| . | . |
| . | . |
| a12 | 48.59 |
| b12 | −262.07 |
| Height | 175 |
| Weight | 70 |
| Age | 28 |
| Gender | Male |
| Systolic blood pressure | 100 |
| Diastolic blood pressure | 70 |
| Pulse | 70 |

As shown in FIG. 5, in this embodiment, after FFT is performed on the signals, the real parts and the imaginary parts (i.e., the plurality of extracted characteristics) of the respective prior twelve (12) harmonic waves of each of the twelve (12) infrared single pulse signals are obtained. One set of results is selected and applied in Formula 4 to rebuild the waveform. It is therefore verified that a similar waveform can be rebuilt by only selecting the prior twelve (12) harmonic waves, thus proving that aₖ and bₖ can represent the characteristics of the pulse waves. In this embodiment, the reason for selecting only the real parts and the imaginary parts (i.e., the plurality of extracted characteristics) of the respective prior twelve (12) harmonic waves of each of the twelve (12) waves is to reduce the computation load. The reason is that, if the real parts and the imaginary parts of all harmonic waves of each of the twelve (12) infrared single pulse signals are all inputted into the neural network, the computation data volume will be overwhelming, which will result in difficulty of extracting characteristics and non-convergence of computation results. Therefore, it is preferred that in this embodiment, the selection of only the real parts and the imaginary parts (i.e., the plurality of extracted characteristics) of the respective prior twelve (12) harmonic waves of each of the twelve (12) infrared single pulse signals can help reduce the number of the extracted characteristics being inputted to the neural network. Further, according to the above rebuilding and verification process, as shown in FIG. 6, the waveform characteristics a, b, c, d and e of any infrared single pulse signal can be synthesized by utilizing the sine coefficients and cosine coefficients (i.e., the extracted characteristics) at multiple different frequencies.

Step S4: inputting the plurality of extracted characteristics into a blood pressure measuring model established by a convolutional neural network (CNN) for obtaining a systolic blood pressure value or a diastolic blood pressure value corresponding to the plurality of infrared single pulse signals.

In the invention, the blood pressure measuring model established by a CNN is established and trained according to data obtained from thirty one (31) subjects aged between 20 and 30 over a period of three days. The physiology signal sensor obtains a plurality of infrared physiological signals from the wrist radial artery of each of the subjects; the signal processing mechanism of the invention obtains a plurality of infrared single pulse signals; Fourier expansion is then performed on the twelve (12) independent infrared single pulse signals cut from the plurality of infrared single pulse signals of each subject so as to extract the plurality of extracted characteristics (aₖ and bₖ) of each of the infrared single pulse signal of each subject. The extracted characteristics are the sine coefficients and cosine coefficients obtained from the Fourier expansion and are divided into twelve (12) sets of pulse average values and non-average values corresponding to the same blood pressure value.

Please note that the reason for using the cut infrared single pulse signal as one characteristic is that each heartbeat is an independent event, the infrared single pulse signals look similar but in fact are different from one another, and the human blood pressure value does not change within a short amount of time; as a result, the cut infrared single pulse signal can be used as the training data for the blood pressure measuring model. The training process for the blood pressure measuring model of the invention will first perform normalization so as to normalize the waveform and standard of the inputted infrared single pulse signals of each of the subjects according to the following Formulas 7, 8 and 9. In the abovementioned formulas, k=1, . . . , 12 is the order of the harmonic wave, m is the sequence number of the pulse data, and the capitalized character is the output result after normalization. All pulses a₀ are normalized to 10000, and the pulse a₀ will perform normalization on aₖ and bₖ. The multiplication is performed prior to the division because if the division is performed first, sometimes the small values will be discarded upon computation due to the length of the data type.

$${A}_{k}^{( m )} = {a}_{k}^{( m )} \times 10000 \times \frac{1}{{a}_{0}^{( m )}} \qquad ( Formula 7 )$$

$${B}_{k}^{( m )} = {b}_{k}^{( m )} \times 10000 \times \frac{1}{{a}_{0}^{( m )}} \qquad ( Formula 8 )$$

$${A}_{0}^{( m )} = {a}_{0}^{( m )} \times 10000 \times \frac{1}{{a}_{0}^{( m )}} \qquad ( Formula 9 )$$

As shown in FIG. 7, the blood pressure measuring model comprises steps S41 to S44. Please refer to the following description for details of each of the computation steps of the blood pressure measuring model of the present invention.

Step S41: inputting the plurality of extracted characteristics into a hidden layer formed by a one-dimensional CNN.

The blood pressure measuring model of the invention inputs the plurality of extracted characteristics into a hidden layer formed by a one-dimensional CNN. In this embodiment, the plurality of extracted characteristics have twenty five (25) extracted characteristics including a₀ to a₁₂ and b₁ to b₁₂. After the extracted characteristics are inputted, the one-dimensional CNN is used as the hidden layer.

Step S42: passing through two layers of one-dimensional convolution layers and subsequently passing through a max-pooling computation.

After completing step S41, the extracted characteristics will then pass through two layers of one-dimensional convolution layers. In this embodiment, the number of filters is 100, the kernel size is 10, and the activation function is ReLU. In this embodiment, the reason for adopting one-dimensional convolution as the hidden layer is that it can reach an excellent prediction result after extraction of the one-dimensional time sequence data characteristics. Then the max-pooling computation with its size of 3 is performed.

Step S43: passing through two layers of one-dimensional convolution layers, inputting a pooling layer, and entering a drop out layer.

After completing step S42, the extracted characteristics will then pass through two layers of one-dimensional convolution layers to extract finer characteristics. In this embodiment, the number of the filter is 160 and the kernel size is 10. The pooling layer is then inputted. The pooling layer is not max-pooling but global average pooling. The pooling can average the entire characteristic chart, and the pooling can also avoid overfitting and reduce the output dimension. Prior to inputting the computation result of step S43 into the fully connected layer, the computation result will enter the drop out layer with its ratio of 0.3 in order to further avoid overfitting.

Step S44: entering a fully connected layer for outputting the systolic blood pressure value or the diastolic blood pressure value.

In the end, the fully connected layer will output the systolic blood pressure value and the diastolic blood pressure value of the prediction result of the blood pressure measuring model. After the training of the blood pressure measuring model according to steps S41 to S44, the output result of the blood pressure measuring model is the systolic blood pressure value and the diastolic blood pressure value. The prediction result can be evaluated by means of comparison with the result measured from a commercial blood pressure meter according to the mean difference (MD), standard deviation (SD) and mean absolute deviation (MAD). Please refer to Table 2 provided below, which presents a comparison of two different methods. The result of the cutting method is better than that of the average method. There are no large gaps between any two results; therefore, it is preferred to adopt the cutting data training method. As shown in FIG. 8 and FIG. 9, the x-axis represents the predicted blood pressure values and the y-axis represents the reference blood pressure values; as a result, the difference between the predicted blood pressure values and the reference blood pressure values can be observed.

TABLE 2
Comparison of computation results for
pulse average and pulse non-average

| | 12 pulses on average | | 12 pulses on non-average | |
|---|---|---|---|---|
| | SBP | DBP | SBP | DBP |
| | (mmHg) | (mmHg) | (mmHg) | (mmHg) |
| MD | 2.20 | 3.88 | 2.36 | 1.31 |
| SD | 9.5 | 7.27 | 7.88 | 6.43 |
| MAD | 8.02 | 6.84 | 6.57 | 5.09 |
| MD ± SD | 2.20 ± 9.5 | 3.88 ± 7.27 | 2.36 ± 7.88 | 1.31 ± 6.43 |

According to the blood pressure measuring method and the blood pressure measuring system 1 of the present invention, after noise and respiration signals are filtered out from a plurality of infrared physiological signals 11, a plurality of infrared single pulse signals 12 will be obtained accordingly. Fourier expansion is then performed on each of the plurality of infrared single pulses 12 for extracting characteristics, including sine coefficients and cosine coefficients, of each of the plurality of infrared single pulse signals 12. The extracted characteristics are inputted into a blood pressure measuring model established by a convolutional neural network (CNN) for obtaining an actual blood pressure value corresponding to the plurality of infrared single pulse signals. Such training of the blood pressure measuring model by extracting the characteristics from the infrared single pulse signals does not require a large amount of training data, which means that a small amount of training data can be used. Therefore, it is easier to accomplish the clinical trial of the blood pressure measuring model used in the blood pressure measuring method of the present invention.

Although the present invention has been explained in relation to its preferred embodiments, it is to be understood that many other possible modifications and variations can be made without departing from the spirit and scope of the invention as hereinafter claimed.

What is claimed is:

1. A blood pressure measuring method, used in a blood pressure measuring system, the blood pressure measuring method comprising the following steps:
obtaining a plurality of infrared physiological signals from a wrist radial artery of a user via a physiology signal sensor;
processing the plurality of infrared physiological signals for obtaining a plurality of infrared single pulse signals via a signal processing mechanism;
performing Fourier expansion on each of the plurality of infrared single pulse signals for extracting a characteristic of each of the plurality of infrared single pulse signals; and
inputting the plurality of extracted characteristics into a blood pressure measuring model established by a convolutional neural network (CNN) for obtaining a systolic blood pressure value or a diastolic blood pressure value corresponding to the plurality of infrared single pulse signals.

2. The blood pressure measuring method as claimed in claim 1, wherein the signal processing mechanism comprises the following steps:
utilizing fast Fourier transform (FFT) to confirm a frequency acquisition range of the plurality of infrared physiological signals; and
utilizing a bandpass filter to filter out the plurality of infrared physiological signals outside of the frequency acquisition range.

3. The blood pressure measuring method as claimed in claim 2, wherein the signal processing mechanism comprises the following steps:
marking a waveform valley point of each of the plurality of infrared physiological signals within the frequency acquisition range in order to cut each of the plurality of infrared physiological signals for generating a plurality of infrared single pulse signals; and
calibrating a direct current (dc) level of each of the plurality of infrared single pulse signals to the same level.

4. The blood pressure measuring method as claimed in claim 1, wherein the blood pressure measuring model comprises the following steps:
inputting the plurality of extracted characteristics into a hidden layer formed by a one-dimensional CNN;
passing through two layers of one-dimensional convolution layers and subsequently passing through a max-pooling computation;
passing through two layers of one-dimensional convolution layers, inputting a pooling layer, and entering a drop out layer; and
entering a fully connected layer for outputting the systolic blood pressure value or the diastolic blood pressure value.

5. The blood pressure measuring method as claimed in claim 1, wherein the plurality of extracted characteristics are sine coefficients and cosine coefficients extracted from each of the plurality of infrared single pulse signals after the Fourier expansion.

6. The blood pressure measuring method as claimed in claim 1, wherein the physiology signal sensor is a photoplethysmography sensor (PPG sensor).

7. A blood pressure measuring system, comprising:
a physiology signal sensor, for obtaining a plurality of infrared physiological signals from a wrist radial artery of a user;
a signal processing module, having a signal connection with the physiology signal sensor; the signal processing module is for processing the plurality of infrared physiological signals for obtaining a plurality of infrared single pulse signals via a signal processing mechanism and performing Fourier expansion on each of the plurality of infrared single pulse signals for extracting a characteristic of each of the plurality of infrared single pulse signals; and
a calculation module, having a signal connection with the signal processing module; the calculation module is for obtaining a systolic blood pressure value or a diastolic blood pressure value corresponding to the plurality of infrared physiological signals by inputting the plurality of extracted characteristics into a blood pressure measuring model established by a convolutional neural network (CNN) for obtaining a systolic blood pressure value or a diastolic blood pressure value corresponding to the plurality of infrared single pulse signals.

8. The blood pressure measuring system as claimed in claim 7, wherein the physiology signal sensor is a photoplethysmography sensor (PPG sensor).

9. The blood pressure measuring system as claimed in claim 8, wherein the blood pressure measuring system is installed in a smart wearable device.

10. The blood pressure measuring system as claimed in claim 7, wherein the signal processing mechanism comprises the following steps:
utilizing fast Fourier transform (FFT) to confirm a frequency acquisition range of the plurality of infrared physiological signals; and
utilizing a bandpass filter to filter out the plurality of infrared physiological signals outside of the frequency acquisition range.

11. The blood pressure measuring system as claimed in claim 10, wherein the signal processing mechanism comprises the following steps:
marking a waveform valley point of each of the plurality of infrared physiological signals within the frequency acquisition range in order to cut each of the plurality of infrared physiological signals for generating a plurality of infrared single pulse signals; and
calibrating a direct current (dc) level of each of the plurality of infrared single pulse signals to the same level.

12. The blood pressure measuring system as claimed in claim 7, wherein the blood pressure measuring model comprises the following steps:
inputting the plurality of extracted characteristics into a hidden layer formed by a one-dimensional CNN;
passing through two layers of one-dimensional convolution layers and subsequently passing through a max-pooling computation;
passing through two layers of one-dimensional convolution layers, inputting a pooling layer, and entering a drop out layer; and
entering a fully connected layer for outputting a systolic blood pressure value or a diastolic blood pressure value.

13. The blood pressure measuring system as claimed in claim 7, wherein the plurality of extracted characteristics are sine coefficients and cosine coefficients extracted from each of the plurality of infrared single pulse signals after the Fourier expansion.
